(19) United States
(12) Patent Application Publication
(10) Pub. No.: US 2018/0163260 A1
(43) Pub. Date: Jun. 14, 2018

(54) GENE POLYMORPHISM ANALYSIS DEVICE

(71) Applicant: SHIMADZU CORPORATION, Kyoto-shi (JP)

(72) Inventors: Akira HARADA, Kyoto-shi (JP); Masamitsu SHIKATA, Kyoto-shi (JP)

(73) Assignee: SHIMADZU CORPORATION, Kyoto-shi (JP)

(21) Appl. No.: 15/378,787

(22) Filed: Dec. 14, 2016

(51) Int. Cl. C12Q 1/68

(52) U.S. Cl. CPC C12Q 1/6827; C12Q 1/6818

(57) ABSTRACT

A gene polymorphism analysis device for determining the allele mating type of a gene polymorphism on the basis of the first fluorescence intensity change over time and the second fluorescence intensity change over time measured on the first allele and second allele that constitute a gene polymorphism of a target DNA. The determination of the allele mating type of the gene polymorphism is performed on samples arranged in M rows and N columns, and the determination results and graphs are displayed on a display device. The graphs show the first fluorescence intensity change over time and the second fluorescence intensity change over time of each sample. The determination results and the graphs are arranged in M rows and N columns so as to correspond to the layout including M rows and N columns of the samples.

### TECHNICAL FIELD

The present invention relates to a gene polymorphism analysis device for determining the gene polymorphism of genomic DNA; in particular, the present invention pertains to a gene polymorphism analysis device for determining the gene polymorphism using TaqMan (registered trademark), Invader (registered trademark), Scorpion (registered trademark), Cycleave, SmartAmp (registered trademark), Lamp (registered trademark), light-emitting substances bound to a nucleic acid (SYBR (registered trademark) Green I, EvaGreen, or the like), Molecular Beacon, Allele Specific PCR, or the like.

### BACKGROUND ART

“Gene polymorphism” is an individual difference of the nucleotide sequence of DNA that constitutes a gene, and in general, it is defined to appear at a collective frequency of 1% or greater. In addition to a single nucleotide polymorphism (SNP) in which only a single nucleotide of a DNA nucleotide sequence mutates, there are a microsatellite polymorphism that is a difference in the number of repeats of a single unit of about from 2 bases to 4 bases, deletion or insertion of a base as gene polymorphism. It is known that gene polymorphism influences drug metabolism as well as how a person becomes sick easily, and the base of SNP site is determined to diagnose disease morbidity, to predict the effect of the administered drug, side effects, and so on.

There is a method that uses an Invader reaction as one of the evaluation methods of determining gene polymorphism. In an Invader reaction, for example, used for determining the mating type of a single nucleotide polymorphism is an Invader reaction reagent containing two kinds of FRET probes containing different fluorescent substances corresponding to two kinds of alleles that constitute SNP, Cleavase (registered trademark) which is an enzyme that recognizes and cuts the structure (intrusion structure) having overlapped oligonucleotides, oligonucleotide (Invader oligo) that invades between the allele oligo and the determination target gene hybridized in the determination target gene in the SNP site, and two kinds of oligonucleotides (allele oligo) that recognize the nucleotide sequence of the site (SNP site) in which a single nucleotide polymorphism occurs.

When these Invader reaction reagents are mixed with DNA containing SNP of a determination target to perform an Invader reaction, a fluorescence signal is generated according to the existence of SNP corresponding to each allele oligo. Excess of allele oligo and fret probes are contained in the reaction reagent, and an Invader reaction is repeated by this excess allele oligo, amplifying the fluorescence signal. Therefore, the fluorescence signal intensity detected by a fluorescence detector gradually increases from the start of the reaction, eventually reaching a plateau.

FIG. 3 is a figure illustrating an example of the fluorescence signal intensity change over time obtained by an Invader reaction. The solid line represents the fluorescence signal intensity change over time derived from the first allele, and the dashed line represents the fluorescence signal intensity change over time derived from the second allele. The left vertical axis and the right vertical axis of the graph represent the fluorescence signal intensity (arbitrary unit) of the first allele and the fluorescence signal intensity derived from the second allele (arbitrary unit), respectively. The horizontal axis represents the elapsed time (reaction time: second) after the start of the Invader reaction, i.e., after setting to the temperature required for the Invader reaction.

Thereby, the existence of SNP corresponding to allele oligo, and whether that SNP is a homozygous or heterozygous are determined based on the fluorescence signal intensity at the time a specific time has passed since the start of reaction, the time until the fluorescence signal intensity reaches a plateau, the fluorescence signal intensity at the time, and other parameters. (For example, refer to Patent Literature 1 through Patent Literature 3).

Such gene polymorphism is determined with many samples, for example, a plurality of wells in which samples are arranged in 8 rows and 12 columns (M rows and N columns) are used.

And the results of the determination of gene polymorphism using the wells are displayed on a display device as an image and presented to a medical practitioner, and others. FIG. 4 is a figure showing an example of a screen displaying 384 determination results by a conventional gene polymorphism analysis device. On the upper half screen, 384 cells are arranged and displayed so as to become the same 16 rows and 24 columns as the sample arrangement of the wells. In each cell, one diamond mark is displayed, respectively, and this diamond mark becomes red when the determination result is “allele 1,” green when the determination result is “allele 1 & 2,” blue when the determination result is “allele 2,” purple when the determination result is “unknown,” and gray when the determination result is “NC.” Displayed on the lower half screen are the sample name, the fluorescence intensity 1, the fluorescence intensity 2, the determination, and comments in 384 rows so as to line up in one row.

FIG. 5 is a figure showing an example of a screen that can display 96 determination results by a conventional gene polymorphism analysis device. On the right half screen, the measurement information is arranged and displayed so as to become the same 8 rows and 12 columns as that of the sample arrangement of the wells. On the left half screen, circles showing the samples selected using an input device on the right half screen are displayed on the coordinates, where the horizontal axis shows the fluorescence signal intensity of the first allele at 40 cycles, and the vertical axis shows the fluorescence signal intensity derived from the second allele at 40 cycles. The circles on these coordinates become green when the determination result is “allele 1 & 2,” red when the determination result is “allele 1,” orange when the determination result is “allele 2,” light blue when the determination result is “NTC,” and black when the determination result is “undetermined.” In FIG. 5, 17 samples are chosen, and 17 circles are displayed on the same coordinates.

### PRIOR ART LITERATURES

### Patent Literatures

(Patent Literature 1) Japanese Unexamined Patent Application Publication No. 2002-300894
(Patent Literature 2) International Publication WO 2006/No. 106867 gazette
(Patent Literature 3) Japanese Unexamined Patent Application Publication No. 2010-104360

### SUMMARY OF THE INVENTION

### Problem to be Solved by the Invention

Incidentally, when a medical practitioner, or others, determines whether the determination result is appropriate or not, he/she usually checks the waveform data (the fluorescence signal intensity change over time) as shown in FIG. 3 used for the determination; however, in the gene polymorphism analysis device mentioned above, the waveform data is displayed on a screen other than the screen shown in FIG. 4 or FIG. 5. For example, the waveform data of the samples corresponding to the diamond marks is displayed on another screen by choosing, using an input device, one diamond mark from among 384 diamond marks in the upper half screen shown in FIG. 4. Therefore, a task of matching the display content of one screen and the display contents of two screens occurs, causing the task to become complicated, which is problematic.

Thus, the objective of the present invention is to provide a gene polymorphism analysis device with which the determination results and the accuracy of the determination results can be easily grasped.

### Means for Solving the Problem

The gene polymorphism analysis device of the present invention made in order to solve the aforementioned problem is a gene polymorphism analysis device for determining an allele mating type of gene polymorphism based on a first fluorescence intensity change over time and a second fluorescence intensity change over time respectively measured on a first allele and a second allele that constitute the gene polymorphism of the target DNA. The gene polymorphism analysis device is equipped with a display device; an analyzing unit for determining the allele mating type of gene polymorphism of samples arranged in a layout including M rows and N columns; and a data display control unit for displaying on the display device determination results determination results of the allele mating type of gene polymorphism and graphs showing the first fluorescence intensity change over time and the second fluorescence intensity change over time of each sample, determination results and the graphs arranged in M rows and N columns so as to correspond to the layout including M rows and N columns of the samples.

The “M rows and N columns” here refer to a sample layout method for registering the measurement information, wherein any layout method is chosen by a measurer, for example, in a plurality of wells used for determining gene polymorphism. Therefore, at least either M or N may be a positive integer of two or greater, for example, 8 rows and 12 columns, 1 row and 2 columns, 2 rows and 1 column, and so on.

### Effect of the Invention

As mentioned above, according to the gene polymorphism analysis device of the present invention, since the fluorescence intensity change over time (waveform data) can be checked along with the determination results on the same screen, the validity of the determination result can be checked at a glance.

### BRIEF DESCRIPTION OF THE DRAWINGS

FIG. 1 is a figure showing an example of a schematic structure of a gene polymorphism analysis device that is one embodiment of the present invention.

FIG. 2 is a figure showing an example of a display screen showing 96 determination results obtained by the device of FIG. 1.

FIG. 3 is a figure showing an example of the fluorescence signal intensity change over time obtained by an Invader reaction.

FIG. 4 is a figure showing an example of a display screen showing 384 determination results obtained by a conventional gene polymorphism analysis device.

FIG. 5 is a figure showing an example of a display screen showing 96 determination results obtained by a conventional gene polymorphism analysis device.

### DETAILED DESCRIPTION OF THE EXEMPLARY EMBODIMENTS

Examples of embodiment of the present invention will be described below using the drawings. The present invention is not limited to the examples of embodiment described below; various modifications are included in the range that does not deviate from the gist of the present invention.

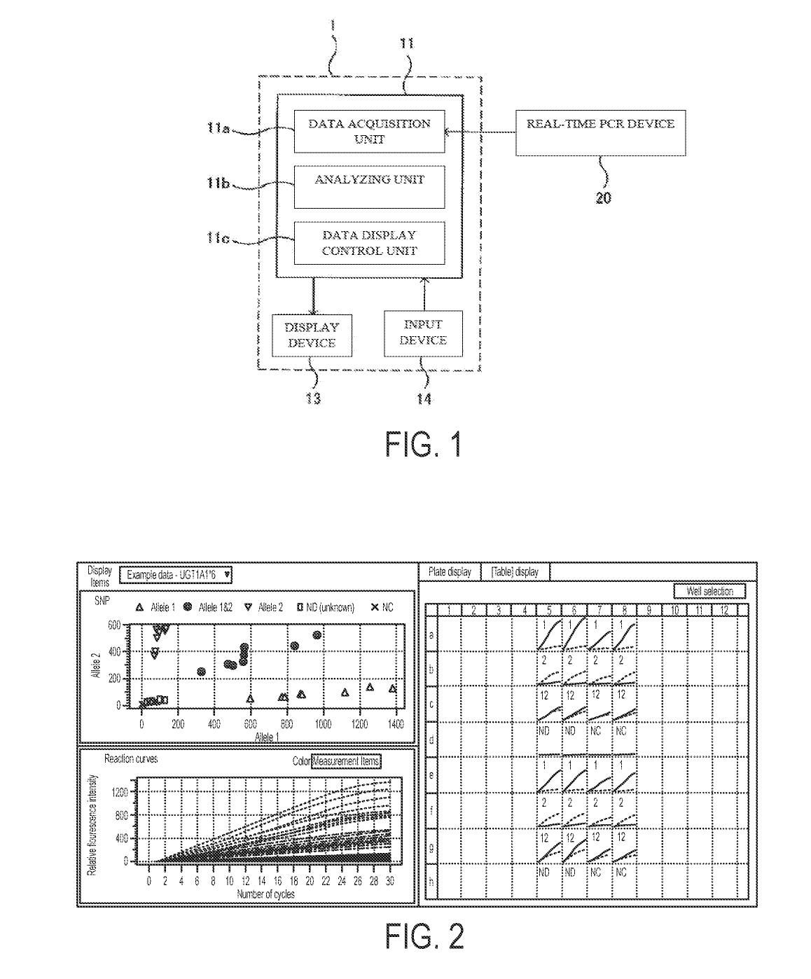

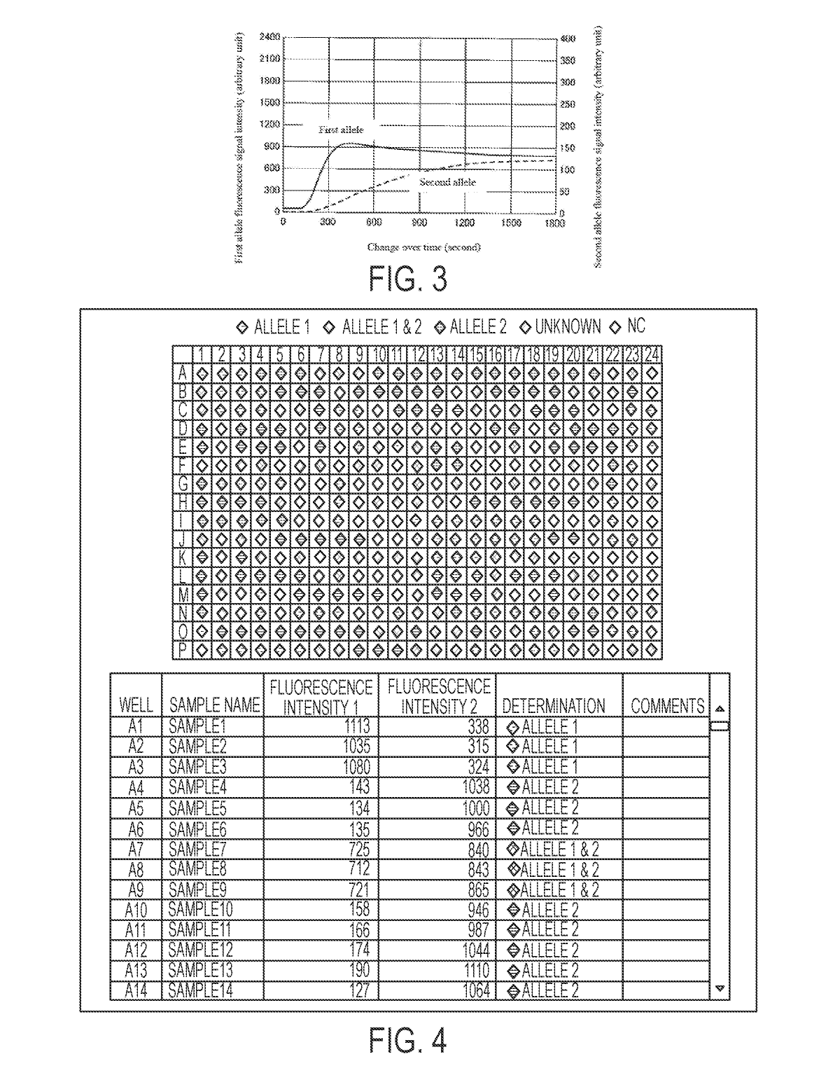

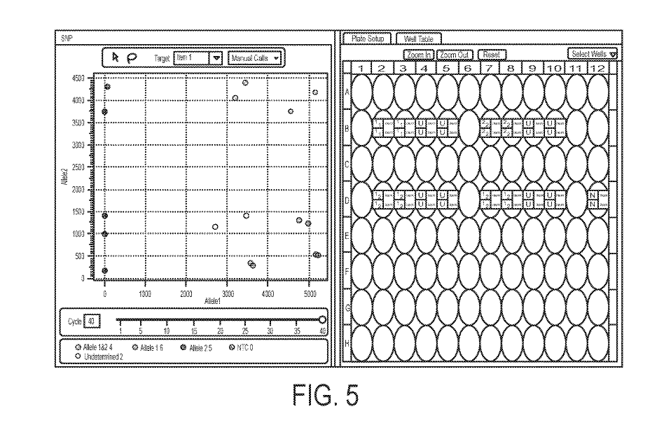

FIG. 1 is a figure illustrating an example of a schematic structure of the gene polymorphism analysis device that is one embodiment of the present invention. The gene polymorphism analysis device 1 is connected to a real-time PCR device 20, equipped with CPU 11, and connected to a display device 13 having a monitor display, or the like, and an input device 14 having a keyboard, a mouse, and the like.

To describe the blocking of a function that CPU 11 processes, the following units are included: a data acquisition unit 11a for acquiring data from the real-time PCR device 20, an analyzing unit 11b for analyzing the data acquired by the data acquisition unit 11a, and a data display control unit 11c for displaying the determination result analyzed by the analyzing unit 11b on the display device 13. Here, the case of acquiring data using a plurality of wells in which samples are arranged in 8 rows and 12 columns (M rows and N columns) will be described.

The analyzing unit 11b performs control for analyzing the data acquired by the data acquisition unit 11a. For example, the existence of SNP corresponding to allele oligo, and whether that SNP is a homozygous or heterozygous are determined based on the fluorescence signal intensity derived from the first allele and the fluorescence signal intensity derived from the second allele at the time a specific time since the reaction start has passed, the time until the fluorescence signal intensity derived from the first allele and the fluorescence signal intensity derived from the second allele reaches a plateau, and the fluorescence signal intensity derived from the first allele and the fluorescence signal intensity derived from the second allele at the time, and other parameters.

The data display control unit 11c controls the display of the following display units on the display device 13: SNP plotted display unit (SNP image) for showing the fluorescence signal intensity of the first allele by the horizontal axis and the fluorescence signal intensity derived from the second allele by the vertical axis, a waveform data display unit (reaction curve image) for displaying in piles a plurality of waveform data (reaction curves) illustrating the fluorescence signal intensity change over time after the start of measurement, and a display unit (graph image) in which the determination result and the waveform data are individually arranged so as to become the same 8 rows 12 columns as that of the sample arrangement of a plurality of wells. FIG. 2 is a figure showing an example of a screen on which 32 determination results are displayed by the gene polymorphism analysis device 1. On the right half screen (graph image), 96 cells are arranged and displayed so as to become the same 8 rows and 12 columns as that of the sample arrangement of a plurality of wells. And one waveform data is displayed in a 4×8 cell into which the measurement information has been registered, respectively. As for the waveform data, the fluorescence signal intensity change over time derived from the first allele is shown by a solid line on the same coordinates, and the fluorescence signal intensity change over time derived from the second allele is shown by a dashed line. The vertical axis of the coordinates shows the fluorescence signal intensity (arbitrary unit), and the horizontal axis shows the elapsed time (the number of measurement cycles) after the start of measurement. The minimum and maximum values of the vertical axis and the horizontal axis are the same in all wells and in the initial display state of the waveform data display unit (reaction curve image) on the lower left screen. At the upper left of the waveform data, number “1” is displayed when the determination result is “allele 1,” number “12” is displayed when the determination result is “allele 1 & 2,” number “2” is displayed when the determination result is “allele 2,” “ND” is displayed when the determination result is “unknown,” and “NC” is displayed when the determination result is “NC.”

On the upper left screen (SNP image), markings showing the samples selected on the right half screen are displayed on coordinates where the horizontal axis represents the fluorescence signal intensity of the first allele at the time of a determination cycle (showing the time a specific time has passed from the start of the reaction: 30 cycles) and the vertical axis represents the fluorescence signal intensity derived from the second allele at the time of a determination cycle (showing the time a specific time has passed from the start of the reaction: 30 cycles). The marking on this coordinate turns into an upward triangle when the determination result is “allele 1,” a double circle when the determination result is “allele 1 & 2,” a downward triangle when the determination result is “allele 2,” a square when the determination result is “unknown,” and x when the determination result is “NC.” And in FIG. 2, 32 markings are displayed on the same coordinates.

Furthermore, on the lower left screen (reaction curve image), a plurality of waveform data (reaction curve) (for example, 4×8 samples) is displayed in piles, where the horizontal axis represents the elapsed time (the number of measurement cycles) after the start of measurement and the vertical axis represents the fluorescence signal intensity (arbitrary unit). That is, in FIG. 2, 32 samples are chosen, and the fluorescence signal intensity change over time derived from the first allele in 32 samples and the fluorescence signal intensity change over time derived from the second allele in 32 samples are displayed on the same coordinates.

According to the gene polymorphism analysis device 1 of the present invention, the fluorescence intensity change over time (waveform data) can be checked along with the determination result on the same screen as shown in FIG. 2; therefore, the validity of the determination result can be checked at a glance.

### Other Examples of Embodiment

(1) The gene polymorphism analysis device 1 mentioned above has a configuration of using a plurality of wells in which samples are arranged in 8 rows and 12 columns; however, a configuration using a plurality of wells in which samples are arranged in 16 rows and 24 columns may also be employed. That is, the number of samples can be set into any number.

(2) The gene polymorphism analysis device 1 mentioned above has a configuration in which the data display control unit 11c displays an SNP image, a reaction curve image, and a graph image; however, a configuration in which at least one image chosen from among the SNP image, the reaction curve image, and the graph image is displayed may also be employed.

(3) In the gene polymorphism analysis device 1 mentioned above, the data display control unit 11c was configured to display SNP plotted display unit (SNP image) for showing the fluorescence signal intensity of the first allele by the horizontal axis and the fluorescence signal intensity derived from the second allele by the vertical axis, a waveform data display unit (reaction curve image) for displaying in piles a plurality of waveform data (reaction curves) illustrating the fluorescence signal intensity change over time after the start of measurement, and a display unit (graph image) in which the determination results and the waveform data are individually arranged so as to become in the same 8 rows and 12 columns as that of the sample arrangement of a plurality of wells; however, it may also be configured by displaying any other display unit (image) containing the display unit (graph image) in which the determination results and the waveform data are individually arranged so as to become in the same 8 rows and 12 columns as that of the sample arrangement of a plurality of wells.

The present invention can be used for the gene polymorphism analysis device for determining gene polymorphism.

### EXPLANATIONS OF REFERENCES

1: Gene polymorphism analysis device
13: Display device

What is claimed is:

1. A gene polymorphism analysis device, for determining an allele mating type of gene polymorphism based on a first fluorescence intensity change over time and a second fluorescence intensity change over time respectively measured on a first allele and a second allele that constitute the gene polymorphism of the target DNA, comprising:
a display device;
an analyzing unit for determining the allele mating type of gene polymorphism of samples arranged in a layout including M rows and N columns; and
a data display control unit for displaying on the display device determination results of the allele mating type of gene polymorphism and graphs showing the first fluorescence intensity change over time and the second fluorescence intensity change over time of each sample, the determination results and the graphs arranged in M rows and N columns so as to correspond to the layout including M rows and N columns of the samples.

2. A gene polymorphism analysis method, for determining an allele mating type of gene polymorphism based on a first fluorescence intensity change over time and a second fluorescence intensity change over time respectively measured on a first allele and a second allele that constitute the gene polymorphism of the target DNA, comprising:
determining the allele mating type of gene polymorphism of samples arranged in a layout including M rows and N columns, and
displaying on a display device determination results of the allele mating type of gene polymorphism and graphs showing the first fluorescence intensity change over time and the second fluorescence intensity change over time of each sample, determination results and the graphs arranged in M rows and N columns so as to correspond to the layout including M rows and N columns of the samples.
